(19) United States
(12) Patent Application Publication
(10) Pub. No.: US 2026/0297007 A1
(43) Pub. Date: Oct. 1, 2026

(54) BETA-ALKOXY KETONE HAVING LONG-LASTING FRAGRANCE AND PREPARATION METHOD AND USE THEREOF

(71) Applicant: Guangzhou Flower Flavours Fragrances Co., Ltd, Guangzhou City (CN)

(72) Inventors: Guoyin LAI, Guangzhou City (CN); Yao WANG, Guangzhou City (CN); Haiming ZOU, Guangzhou City (CN); Xiangping LI, Guangzhou City (CN); Chunning LUO, Guangzhou City (CN); Baojun XIE, Guangzhou City (CN); Xiu WU, Guangzhou City (CN); Minfang XIAO, Guangzhou City (CN)

(21) Appl. No.: 19/400,196

(22) Filed: Nov. 25, 2025

(30) Foreign Application Priority Data
Mar. 27, 2025 (CN) 202510377740.5

(51) Int. Cl. C07C 49/255; C07C 45/64; C07C 45/79

(52) U.S. Cl. CPC C07C 49/255; C07C 45/64; C07C 45/79

(57) ABSTRACT

Disclosed are a β-alkoxy ketone having long-lasting fragrance and a preparation method and use thereof. The preparation method includes: mixing an α,β-unsaturated ketone with an alcohol compound, and conducting a Michael reaction in the presence of an acid catalyst to obtain the β-alkoxy ketone having long-lasting fragrance, where the acid catalyst includes one or more selected from the group consisting of bis(trifluoromethane)sulfonimide, sodium p-toluenesulfinate, tricyclohexyl phosphine, and tetramethylguanidine.

### CROSS REFERENCE TO RELATED APPLICATION

This patent application claims the benefit and priority of Chinese Patent Application No. 202510377740.5 filed with the China National Intellectual Property Administration on Mar. 27, 2025, the disclosure of which is incorporated by reference herein in its entirety as part of the present application.

### TECHNICAL FIELD

The present disclosure belongs to the technical field of organic synthesis, and specifically relates to a β-alkoxy ketone having long-lasting fragrance and a preparation method and use thereof.

### BACKGROUND

α,β-Unsaturated ketones are a class of fragrance molecules widely used in daily life. However, due to the strong volatility of α,β-unsaturated ketones, it is difficult to make the fragrance of α,β-unsaturated ketones last for a long time. How to enhance the stability and sustained release of fragrances to improve the scent retention has become a pivotal challenge in the development of fragrances.

Traditionally, α,β-unsaturated ketones are converted into β-mercapto ketones to achieve the slow release. However, the above conversion process requires the use of malodorous sulfur agents, which typically causes the irritation to individuals. Moreover, β-mercapto ketones release α,β-unsaturated ketones very quickly, resulting in poor fragrance retention.

### SUMMARY

An object of the present disclosure is to provide a β-alkoxy ketone having long-lasting fragrance and a preparation method and use thereof. The β-alkoxy ketone prepared by the method according to the present disclosure could slowly release an α,β-unsaturated ketone, which results in achieving the prolonged fragrance retention.

To achieve the above object, the present disclosure provides the following technical solutions:
The present disclosure provides a method for preparing a β-alkoxy ketone having long-lasting fragrance, including the following step:
mixing a α,β-unsaturated ketone with an alcohol compound, and conducting a Michael reaction in the presence of an acid catalyst to obtain the β-alkoxy ketone having long-lasting fragrance,
where the acid catalyst includes one or more selected from the group consisting of bis(trifluoromethane)sulfonimide, sodium p-toluenesulfinate, tricyclohexyl phosphine, and tetramethylguanidine.

In some embodiments, the α,β-unsaturated ketone has a structure shown in formula I:

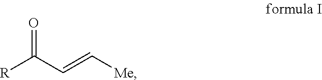

where R includes one selected from the group consisting of alkyl, aryl, amino, and hydroxyl.

In some embodiments, the alcohol compound has a structure shown in formula II:

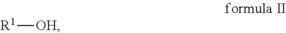

where R¹ includes one selected from the group consisting of alkyl, substituted alkyl, aryl, or benzyl;
the alkyl includes one selected from the group consisting of dodecyl, methyl, and ethyl; and the substituted alkyl includes trifluoroethyl.

In some embodiments, the α,β-unsaturated ketone is delta-damascone; and the alcohol compound is selected from the group consisting of benzyl alcohol, lauryl alcohol, and trifluoroethanol.

In some embodiments, a molar ratio of the α,β-unsaturated ketone to the alcohol compound is in a range of 1:1 to 1:3.

In some embodiments, a molar mass of the acid catalyst is 0.5% to 10% of a molar mass of the α,β-unsaturated ketone.

In some embodiments, the Michael reaction is conducted at room temperature for 2 h to 120 h.

In some embodiments, after the Michael reaction, the method further includes purifying a resulting product by column chromatography.

The present disclosure also provides a β-alkoxy ketone having long-lasting fragrance prepared by the method as described in the above technical solutions.

The present disclosure also provides use of the β-alkoxy ketone having long-lasting fragrance as described in the above technical solutions as a fragrance.

The present disclosure provides a method for preparing a β-alkoxy ketone having long-lasting fragrance, including the following step: mixing a α,β-unsaturated ketone with an alcohol compound, and conducting a Michael reaction in the presence of an acid catalyst to obtain the β-alkoxy ketone, where the acid catalyst includes one or more selected from the group consisting of bis(trifluoromethane)sulfonimide, sodium p-toluenesulfinate, tricyclohexyl phosphine, and tetramethylguanidine. Based on inexpensive and readily-available alcohol raw materials, the present disclosure provides the method for preparing a β-alkoxy ketone that involves simple operations and mild conditions. Since the leaving ability of alkoxy is weaker than the leaving ability of mercapto, the β-alkoxy ketone could release an α,β-unsaturated ketone more slowly than a β-mercapto ketone, which results in achieving the prolonged fragrance retention.

In addition, the present disclosure does not require heating or cooling and an inert atmosphere, allows a reaction to proceed at room temperature in air, and only requires a catalytic amount of an acid. The method provided by the present disclosure could efficiently and highly selectively convert an alcohol and an α,β-unsaturated ketone into a β-alkoxy ketone, with inexpensive and readily-available raw materials, simple operations, and mild reaction conditions. Therefore, the method is suitable for industrial production.

### BRIEF DESCRIPTION OF THE DRAWINGS

FIG. 1 shows a proton nuclear magnetic resonance (¹H-NMR) spectrum of a target product 1;

FIG. 2 shows a gas chromatography-mass spectrometry (GC-MS) spectrum of the target product 1;

FIG. 3 shows a ¹H-NMR spectrum of a target product 2;

FIG. 4 shows a GC-MS spectrum of the target product 2;

FIG. 5 shows a GC-MS spectrum of a target product 3; and

FIG. 6 shows results of fragrance lingering tests for target products in Test Example 1.

### DETAILED DESCRIPTION OF THE EMBODIMENTS

The present disclosure provides a method for preparing a β-alkoxy ketone having long-lasting fragrance, including the following step:
mixing an α,β-unsaturated ketone with an alcohol compound, and conducting a Michael reaction in the presence of an acid catalyst to obtain the β-alkoxy ketone,
where the acid catalyst includes one or more selected from the group consisting of bis(trifluoromethane)sulfonimide, sodium p-toluenesulfinate, tricyclohexyl phosphine, and tetramethylguanidine.

In some embodiments of the present disclosure, the α,β-unsaturated ketone has a structure shown in formula I:

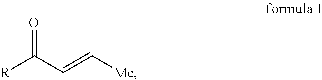

where R preferably includes one selected from the group consisting of alkyl, aryl, amino, and hydroxyl.

In some embodiments of the present disclosure, the α,β-unsaturated ketone is delta-damascone.

In some embodiments of the present disclosure, the alcohol compound has a structure shown in formula II:

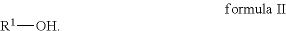

In some embodiments of the present disclosure, R¹ includes one selected from the group consisting of alkyl, substituted alkyl, aryl, and benzyl, where the alkyl preferably includes one selected from the group consisting of dodecyl, methyl, and ethyl. In some embodiments, the substituted alkyl includes trifluoroethyl. In some embodiments of the present disclosure, the alcohol compound is selected from the group consisting of benzyl alcohol, lauryl alcohol, and trifluoroethanol.

In some embodiments of the present disclosure, a molar ratio of the α,β-unsaturated ketone to the alcohol compound is in a range of 1:1 to 1:3. In some embodiments of the present disclosure, the molar ratio of the α,β-unsaturated ketone to the alcohol compound is 1:2.

In the present disclosure, the acid catalyst includes one or more selected from the group consisting of bis(trifluoromethane)sulfonimide, sodium p-toluenesulfinate, tricyclohexyl phosphine, and tetramethylguanidine. In some embodiments of the present disclosure, a molar mass of the acid catalyst is 0.5% to 10% of a molar mass of the α,β-unsaturated ketone. In some embodiments of the present disclosure, the molar mass of the acid catalyst is 0.5%, 1%, 2%, 3%, 4%, 5%, 6%, 7%, 8%, 9%, and 10% of the molar mass of the α,β-unsaturated ketone.

In some embodiments of the present disclosure, the Michael reaction is conducted at room temperature. In some embodiments of the present disclosure, the Michael reaction is conducted for 2 h to 120 h. In some embodiments of the present disclosure, the Michael reaction is conducted for 2 h, 12 h, 24 h, 48 h, 72 h, 96 h, and 120 h. In some embodiments of the present disclosure, after the Michael reaction, the method further includes: purifying a resulting product by column chromatography. In the present disclosure, there are no specific limitations on a process of the column chromatography, and the process of the column chromatography well known to those skilled in the art may be adopted.

A chemical reaction equation for the method according to the present disclosure is as follows (where the catalyst is bis(trifluoromethane)sulfonimide):

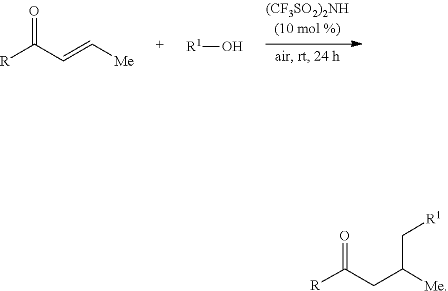

The present disclosure also provides a β-alkoxy ketone prepared by the method as described in the above technical solutions.

In some embodiments of the present disclosure, the β-alkoxy ketone having long-lasting fragrance has the structure shown in formula III:

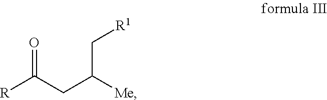

where R preferably includes one selected from the group consisting of alkyl, aryl, amino, and hydroxyl;
R¹ preferably includes one selected from the group consisting of alkyl, substituted alkyl, aryl, and benzyl; the alkyl preferably includes one selected from the group consisting of dodecyl, methyl, and ethyl; and the substituted alkyl preferably includes trifluoroethyl.

The present disclosure also provides use of the β-alkoxy ketone having long-lasting fragrance as described in the above technical solution as a fragrance.

Unless otherwise specified, all materials and devices used in the present disclosure are commercially-available products in the art.

The technical solutions of the present disclosure will be clearly and completely described below with reference to the examples of the present disclosure. Apparently, the described examples are merely some rather than all of the examples of the present disclosure. All other examples obtained by those of ordinary skill in the art based on the examples of the present disclosure without creative efforts shall fall within the scope of the present disclosure.

### Example 1

Benzyl alcohol (1.55 mL, 15 mmol) was mixed with delta-damascone (5.0 mmol), bis(trifluoromethane)sulfonimide (with a molar mass being 10 mol % of a molar mass of the delta-damascone) was added as a catalyst. A resulting mixture was subjected to reaction at room temperature for 24 h to obtain a crude product. The crude product was purified by column chromatography to obtain a target product 1.

A chemical reaction equation was as follows:

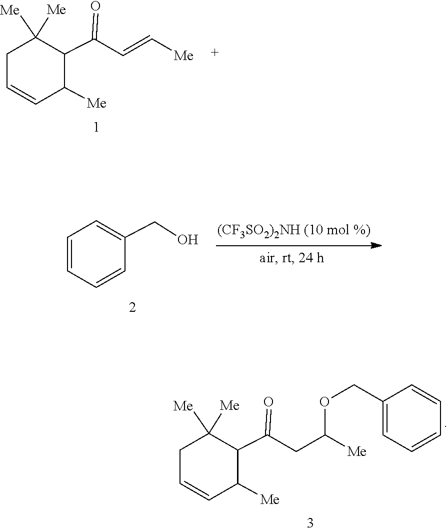 [SMILES: C/C=C/C(=O)C1C(C)C=CCC1(C)C ; OCc1ccccc1 ; CC(CC(=O)C1C(C)C=CCC1(C)C)OCc1ccccc1]

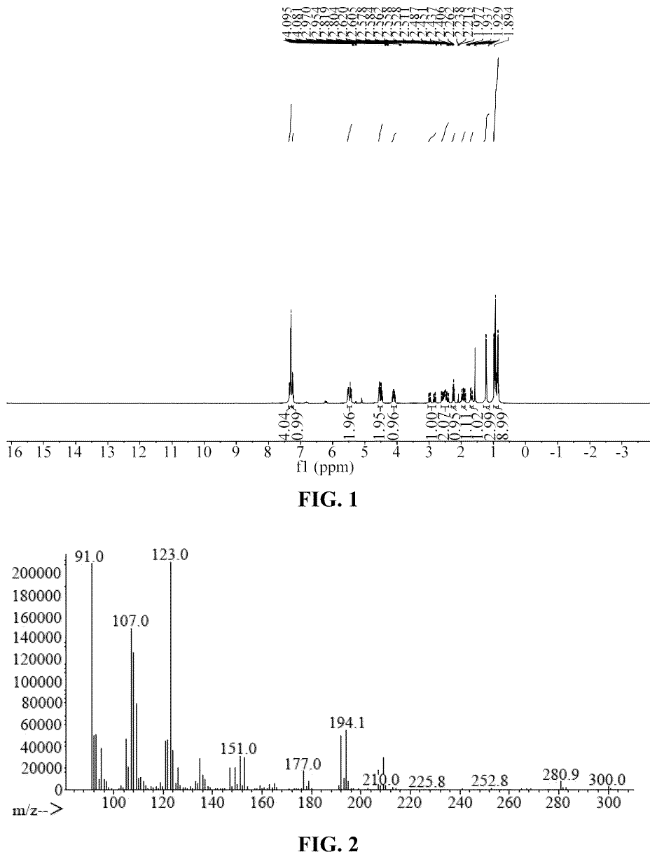

FIG. 1 shows a ¹H-NMR spectrum of the target product 1. FIG. 2 shows a GC-MS spectrum of the target product 1.

It can be seen from FIG. 1 and FIG. 2 that the target product 1 was successfully prepared through the above process.

### Example 2

Lauryl alcohol (3.36 mL, 15 mmol) was mixed with delta-damascone (5.0 mmol), bis(trifluoromethane)sulfonimide (with a molar mass being 10 mol % of a molar mass of the delta-damascone) was added as a catalyst. A resulting mixture was subjected to reaction at room temperature for 24 h to obtain a crude product. The crude product was purified by column chromatography to obtain a target product 2.

A chemical reaction equation was as follows:

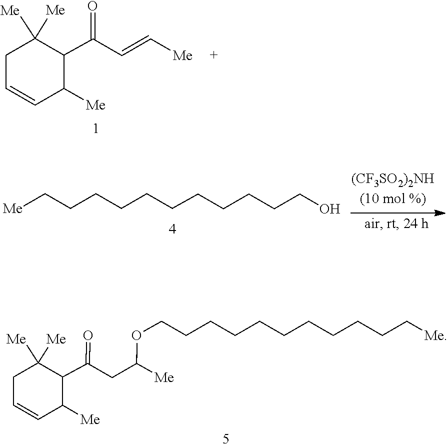 [SMILES: C/C=C/C(=O)C1C(C)C=CCC1(C)C ; CCCCCCCCCCCCO ; CCCCCCCCCCCCOC(C)CC(=O)C1C(C)C=CCC1(C)C]

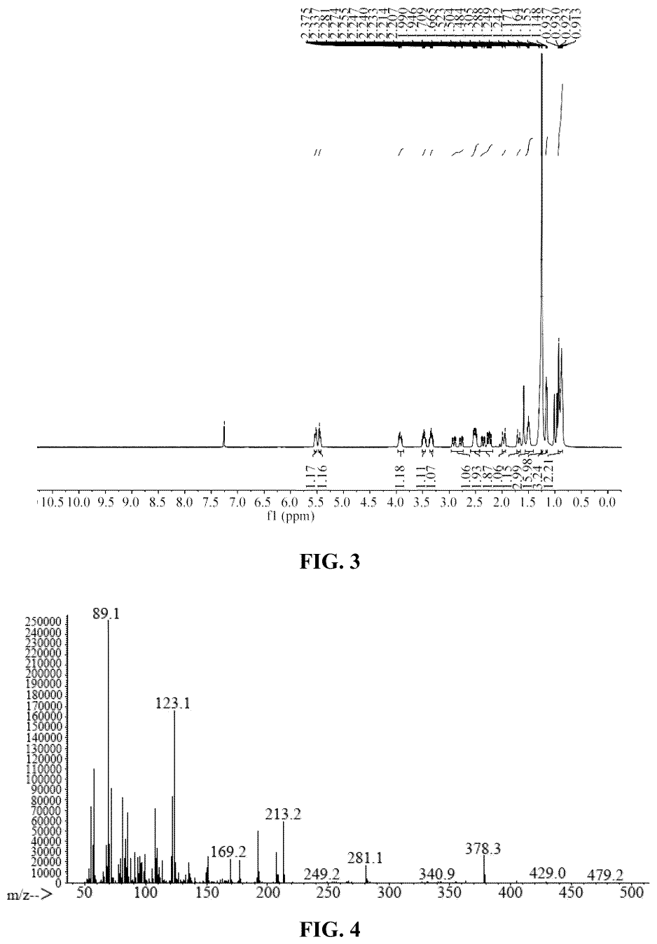

FIG. 3 shows a ¹H-NMR spectrum of the target product 2. FIG. 4 shows a GC-MS spectrum of the target product 2.

It can be seen from FIG. 3 and FIG. 4 that the target product 2 was successfully prepared through the above process.

### Example 3

Trifluoroethanol (1.08 mL, 15 mmol) was mixed with delta-damascone (5.0 mmol), bis(trifluoromethane)sulfonimide (with a molar mass being 10 mol % of a molar mass of the delta-damascone) was added as a catalyst. A resulting mixture was subjected to reaction at room temperature for 24 h to obtain a crude product. The crude product was purified by column chromatography to obtain a target product 3.

A chemical reaction equation was as follows:

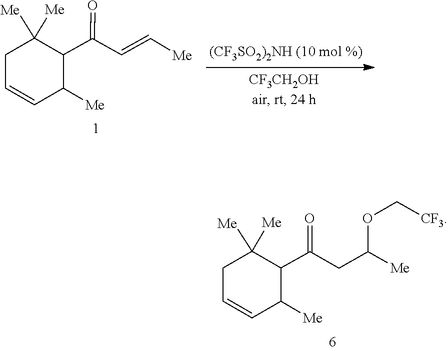 [SMILES: C/C=C/C(=O)C1C(C)C=CCC1(C)C ; CC(CC(=O)C1C(C)C=CCC1(C)C)OCC(F)(F)F]

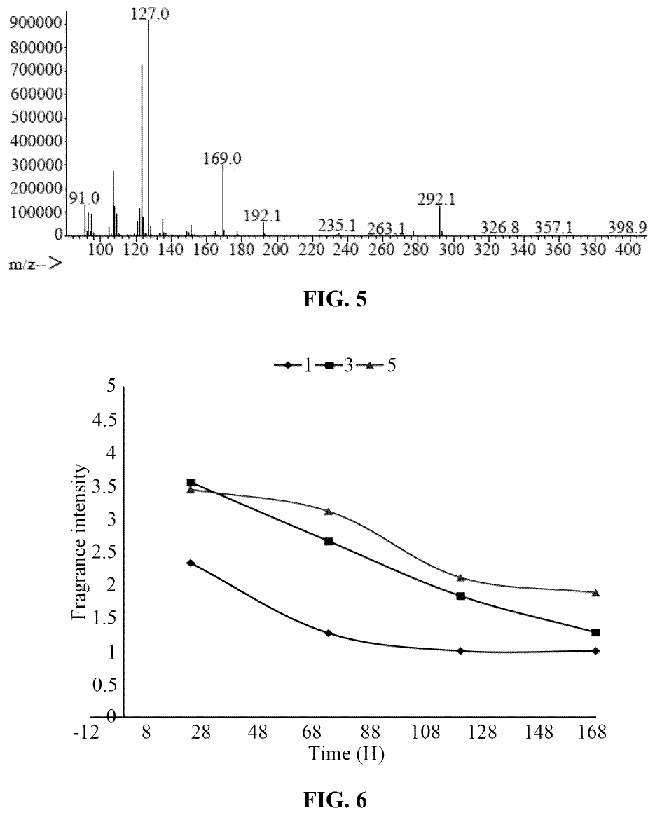

FIG. 5 shows a GC-MS spectrum of the target product 3.

It can be seen from FIG. 5 that the target product 3 was successfully prepared through the above process.

### Performance Testing

Target products prepared in Examples 1 and 2 were used as fragrance samples (target product 1 prepared in Example 1 corresponded to a compound 3 and target product 2 prepared in Example 2 corresponded to a compound 5), and their fragrance lingering effects were tested.

### Test Method:

1 mmol of a fragrance sample was added to 80 g of a fragrance-free liquid laundry matrix. A resulting material was subjected to mixing evenly by vigorously stirring to obtain a mixture. The mixture was poured into a “Midea MB55V35E” domestic washing machine filled with 60 L of tap water, and 40 small towels (30*30 cm and about 40 g each) were subjected to washing under a standard program at room temperature. After the washing was completed, 40 washed small towels were dried in a drying chamber for 24 h. 18 dried small towels were randomly selected and then loosely wrapped in aluminum foils. The fragrance evaluation was conducted at 24 h, 72 h, 120 h, and 168 h.

With 18 evaluators, the tested towels were scored based on an intensity scale from 1 to 7 (1 refers to no fragrance, 2 refers to faint fragrance, 3 refers to slightly faint fragrance, 4 refers to moderate fragrance, 5 refers to slightly strong fragrance, 6 refers to strong fragrance, and 7 refers to very strong fragrance).

The same test was conducted with delta-damascone (corresponding to a compound 1) as a comparative sample, and test results are shown in Table 1 and FIG. 6.

TABLE 1
Fragrance lingering effects of the target
products obtained in the examples

| | Mass percentage | Average fragrance intensities | | | |
|---|---|---|---|---|---|
| | of a compound | during different time periods | | | |
| Compound | to the matrix | 24 h | 72 h | 120 h | 168 h |
| 1 | 0.24 | 2.33 | 1.27 | 1 | 1 |
| 3 | 0.38 | 3.55 | 2.66 | 1.83 | 1.28 |
| 5 | 0.47 | 3.44 | 3.11 | 2.11 | 1.88 |

It can be seen from Table 1 and FIG. 6 that the compound 3 and the compound 5 each exhibit a larger average fragrance intensity score than the compound 1 at each time point. The average fragrance intensity score of the compound 1 is “1” (no fragrance) at 120 h, but the compound 3 and the compound 5 are still fragrant at 168 h, indicating that the compound 3 and the compound 5 could release α,β-unsaturated ketones more slowly than the compound 1, which results in achieving the prolonged fragrance retention.

Although the present disclosure has been described in detail through the above embodiments, the embodiments are merely some rather than all of the embodiments of the present disclosure. Other embodiments can be acquired based on these embodiments without creative effort, all of which shall fall within the scope of the present disclosure.

What is claimed is:

1. A method for preparing a β-alkoxy ketone having long-lasting fragrance, comprising the following step:
mixing an α,β-unsaturated ketone with an alcohol compound, and conducting a Michael reaction in the presence of an acid catalyst to obtain the β-alkoxy ketone having long-lasting fragrance,
wherein the acid catalyst comprises one or more selected from the group consisting of bis(trifluoromethane)sulfonimide, sodium p-toluenesulfinate, tricyclohexyl phosphine, and tetramethylguanidine.

2. The method of claim 1, wherein the α,β-unsaturated ketone has a structure shown in formula I: 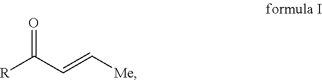
wherein R comprises one selected from the group consisting of alkyl, aryl, amino, and hydroxyl.

3. The method of claim 1, wherein the alcohol compound has a structure shown in formula II: 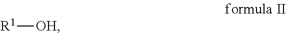
wherein R¹ comprises one selected from the group consisting of alkyl, substituted alkyl, aryl, and benzyl;
the alkyl comprises one selected from the group consisting of dodecyl, methyl, and ethyl; and the substituted alkyl comprises trifluoroethyl.

4. The method of claim 1, wherein the α,β-unsaturated ketone is delta-damascone; and the alcohol compound is selected from the group consisting of benzyl alcohol, lauryl alcohol, and trifluoroethanol.

5. The method of claim 1, wherein a molar ratio of the α,β-unsaturated ketone to the alcohol compound is in a range of 1:1 to 1:3.

6. The method of claim 2, wherein a molar ratio of the α,β-unsaturated ketone to the alcohol compound is in a range of 1:1 to 1:3.

7. The method of claim 3, wherein a molar ratio of the α,β-unsaturated ketone to the alcohol compound is in a range of 1:1 to 1:3.

8. The method of claim 4, wherein a molar ratio of the α,β-unsaturated ketone to the alcohol compound is in a range of 1:1 to 1:3.

9. The method of claim 5, wherein a molar mass of the acid catalyst is 0.5% to 10% of a molar mass of the α,β-unsaturated ketone.

10. The method of claim 1, wherein the Michael reaction is conducted at room temperature for 2 hours (h) to 120 h.

11. The method of claim 1, wherein after the Michael reaction, the method further comprises purifying a resulting product by column chromatography.

12. A β-alkoxy ketone having long-lasting fragrance prepared by the method of claim 1.

13. The β-alkoxy ketone of claim 12, wherein the α,β-unsaturated ketone has a structure shown in formula I: 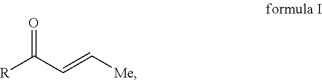
wherein R comprises one selected from the group consisting of alkyl, aryl, amino, and hydroxyl.

14. The β-alkoxy ketone of claim 12, wherein the alcohol compound has a structure shown in formula II: 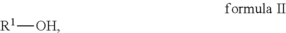
wherein R¹ comprises one selected from the group consisting of alkyl, substituted alkyl, aryl, and benzyl;
the alkyl comprises one selected from the group consisting of dodecyl, methyl, and ethyl; and the substituted alkyl comprises trifluoroethyl.

15. The β-alkoxy ketone of claim 12, wherein the α,β-unsaturated ketone is delta-damascone;
and the alcohol compound is selected from the group consisting of benzyl alcohol, lauryl alcohol, and trifluoroethanol.

16. The β-alkoxy ketone of claim 12, wherein a molar ratio of the α,β-unsaturated ketone to the alcohol compound is in a range of 1:1 to 1:3.

17. The β-alkoxy ketone of claim 12, wherein a molar mass of the acid catalyst is 0.5% to 10% of a molar mass of the α,β-unsaturated ketone.

18. The β-alkoxy ketone of claim 12, wherein the Michael reaction is conducted at room temperature for 2 hours (h) to 120 h.

19. The β-alkoxy ketone of claim 12, wherein after the Michael reaction, the method further comprises purifying a resulting product by column chromatography.
